(19) United States
(12) Patent Application Publication
(10) Pub. No.: US 2013/0064900 A1
(43) Pub. Date: Mar. 14, 2013

(54) Anti-dandruff methods, processes and compositions

(71) Applicants: Bert W. Herring, Jacksonville, FL (US); Judi C. Herring, Jacksonville, FL (US)

(72) Inventors: Bert W. Herring, Jacksonville, FL (US); Judi C. Herring, Jacksonville, FL (US)

(21) Appl. No.: 13/232,939

(22) Filed: Sep. 14, 2011

(51) Int. Cl. A61K 33/24

(52) U.S. Cl. 424/646

(57) ABSTRACT

New methods, compositions, and processes for the treatment of symptoms of dandruff using cellular adhesion modulators are disclosed.

### TECHNICAL FIELD

The invention relates to methods, processes and compositions related to the treatment of symptoms associated with dandruff and other disorders of desquamation.

### BACKGROUND OF THE INVENTION

In general, desquamation is the process of shedding flat, dead cells from the outermost layer of skin including the scalp. Usually the process releases the cells in particles too small (<0.1 mm in diameter) to be readily visible to the naked eye when present on clothing; these low-density particles are readily dispersed by air movement and tend not to accumulate in visible densities. In some situations, such as in bright sunlight, the shed cells may be visible as dust particles. When the desquamation process releases cells in clusters large enough to be readily visible or heavy enough to accumulate on clothing rather than be dispersed by air movement, a significant and often stigmatizing cosmetic concern is created.

In 1874, Louis-Charles Malassez postulated that the presence of a fungus on the scalp was the cause of dandruff. The currently prevailing hypothesis builds on the Malassez theory, adding two more elements required for a person to produce dandruff: a person's normal production of sebum and an individual sensitivity to the waste products of the metabolism of the sebum by the fungus (DeAngelis et al, 2005).

Existing antidandruff treatments seek to reduce or eliminate dandruff formation by targeting one or more aspects of the sebum-fungus-sensitivity triad. The disclosures of antifungal and cytotoxic pyridinethione zinc shampoo are set forth in U.S. Pat. Nos. 2,809,971 and 3,236,733, the disclosures of which are incorporated herein in their entireties by this reference. Alternative antifungal formulations are set forth in U.S. Pat. No. 4,335,125, the disclosure of which is incorporated herein in its entirety by this reference. The most commercially successful antidandruff preparations use antifungal agents such as ketoconazole or zinc pyrithione in a shampoo base formulated to target the resident fungus.

Current treatments do not fully eradicate the symptoms of dandruff in all cases, and none cure the condition. Indeed, dandruff remains a significant focus of product development today. Significantly, current methods address treatment of dandruff by either directly or indirectly targeting the presence of the fungus. Given the fact that the fungus was first identified as the probable cause of dandruff as early as 1874 and anti-fungal shampoos have existed at least since the 1960s, it would be ground-breaking to identify an alternate way to treat dandruff. Moreover, it would be a significant improvement if there were a way to treat dandruff that provided effective treatment using ingredients with effectiveness at low concentrations that were not toxic to humans or other animals.

### SUMMARY OF THE INVENTION

The present invention relates to the use of dandruff-disrupting adhesion modulators for the reduction of dandruff symptoms. Vanadate, pervanadate and other modulators of intercellular adhesion described in the scientific literature have been shown to have positive and negative effects on intercellular adhesion, but the net effect of the adhesion modulators on the complex symbiotic milieu of dandruff has not been recognized outside of the present invention. While vanadate and pervanadate have been extensively studied and used in cellular biology, such use is in the context of either concentrated solutions or diluted in isotonic solutions comprising other solutes that create tonicity sufficient to prevent cell lysis. Disclosure of utility for a solution comprising an adhesion modulator that would be unsuitable and often lethal for use on cells in culture due to very low tonicity or the presence of other agents (for example, preservatives, surfactants) is both novel and surprising. As will be described, the inventive methods, processes and compositions offer significant advantages in effectiveness, safety, and environmental responsibility.

The present invention comprises methods, processes and compostions for reducing the symptoms of dandruff. Possible embodiments of the invention include the incorporation and application of the adhesion modulator in a leave- on, a shampoo, a conditioner or other hair or skin preparation. Another aspect of the invention includes the process of placing an adhesion modulator in a container suitable for dispensing or applying the appropriate amount of an adhesion modulator for convenient personal use and/or application to the skin. The inventive composition includes enhancements comprising the combination of an adhesion modulator and non-exclusive alternatives including a fragrance, a preservative, a skin sensate, a surfactant or combinations thereof. The present invention further specifies the use of adhesion modulators in the manufacture of a medicament for the treatment of dandruff.

Other features and advantages of the invention will be apparent from the detailed description, the preferred embodiments and from the claims. Additional advantages of the invention will be set forth in part in the detailed description which follows and in part will be obvious from the description, or may be learned from the preferred embodiments or by practice of the invention. The advantages of the invention will be realized and attained by means of the elements and combinations particularly pointed out in the appended claims. It is to be understood that both the foregoing general description and the following detailed description are exemplary and explanatory aspects of the invention and are not restrictive of the invention, as claimed.

### DETAILED DESCRIPTION OF THE INVENTION

The present invention may be understood more readily by reference to the following detailed description of the invention and the examples provided therein. It is to be understood that this invention is not limited to the specific methods, formulations and conditions described herein, which, of course, may vary. It is also to be understood that the terminology used herein is for the purpose of describing particular aspects only and is not intended to be limiting.

In this specification and in the claims that follow, reference will be made to a number of terms, which shall be defined to have the following meanings. The singular forms “a,” “an,” and “the” include plural referents unless the context clearly dictates otherwise. As used herein, all percentages are by weight unless otherwise specified. Ranges may be expressed herein as from “about” one particular value and/or to “about” or another particular value. When such a range is expressed, another aspect includes from the one particular value and/or to the other particular value. Similarly, when values are expressed as approximations by use of the antecedent “about,” it will be understood that the particular value forms another aspect.

“Optional” or “optionally” means that the subsequently described event or circumstance may or may not occur, and that the description includes instances where said event or circumstance occurs and instances where it does not. For example, the phrase “optionally comprising water” means that the composition may comprise water and that the description includes both compositions comprising water and compositions without water.

As used herein, the term “pharmaceutically-acceptable” means that drugs, solvents, solutions, medicaments, or inert ingredients that the term describes are suitable for use in contact with the skin of humans and other animals without undue toxicity, incompatibility, irritation or allergic response and have a reasonable risk/benefit ratio.

Throughout this application, where patents are referenced, the disclosures of these patents in their entireties are hereby incorporated by reference into this disclosure.

As used herein, “symptom of dandruff” means one or more of the characteristics that distinguish one who has dandruff from one who does not have dandruff. Non-limiting examples of symptoms of dandruff include shedding of clusters of skin cells in flakes of sufficient size to be readily visible to the unaided eye, the size of the shed flakes, the number of shed flakes, the rate of shedding of flakes, the cosmetic consequences of shed flakes, itching, erythema, and the rate of hair loss.

As used herein, the term “leave-on” and “leave-on treatment” mean a composition that is typically applied to the desired area of skin without subsequently rinsing, shampooing, or otherwise intentionally removing the composition from the area for a period of at least one hour after application. As used herein, the term “leave-on” is to be distinguished from compositions such as shampoos and other similar compositions that are applied to the desired area of the body and then rinsed away within several minutes after application. As used herein, the term “leave-on” includes compositions having one or more components, which, under typical circumstances of use, evaporate from the surface to which they are applied.

As used herein, “topical application” means directly laying on the skin or spreading on the skin, or causing to come in contact with the skin of a person or animal. In some aspects, topical application of the formulations herein may result in some absorption into at least the outer layer of a user's skin.

As used herein, “vanadyl” means vanadium in its +4 oxidation state combined with oxygen, known as the vanadyl ion, VO²⁺.

As used herein, “vanadate” means vanadium in its +5 oxidation state combined with oxygen. The term “vanadate” includes VO₄³⁻ (orthovanadate) and VO₃⁻ (metavanadate), polymeric forms and protonated forms in solution (HVO₄⁻², H₂VO₄⁻).

As used herein, “molybdate” means molybdenum in its +6 oxidation state combined with oxygen. The term “molybdate” includes MO₄⁻² and polymeric forms thereof.

As used herein, “tungstate” means tungsten in its +5 oxidation state combined with oxygen. The term “tungstate” includes WO₄⁻³.

As used herein, “pervanadate” means a compound in which one or more peroxide ions (O₂²⁻) coordinate with vanadium; the term is synonymous with “peroxyvanadate” and “peroxovanadate.”

As used herein, “permolybdate” means a compound in which one or more peroxide ions coordinate with molybdenum; the term is synonymous with “peroxymolybdate” and “peroxomolybdate.”

As used herein, “pertungstate” means a compound in which one or more peroxide ions coordinate with tungsten; the term is synonymous with “peroxytungstate” and “peroxotungstate.”

As used herein, “peroxide” means a compound having an oxygen-oxygen single bond able to combine with an oxometalate. Non-limiting examples of peroxides include hydrogen peroxide, benzoyl peroxide, peracetic acid, and ethyl peroxide.

As used herein, “oxidizer” and “oxidizing agent” mean a compound that readily transfers one or more oxygen atoms to another compound or readily accepts electrons. Non-limiting examples of oxidizers include hydrogen peroxide, chlorine, iodine, chlorate, perchlorate and hypochlorite.

As used herein, “oxometalate” means salts or compounds comprising ionic chemical groups of a metal atom ligated to at least one oxygen atom. The term includes but is not limited to vanadate, molybdate, and tungstate and includes the peroxometalates as described below.

As used herein, “peroxometalate” means a compound or ion comprising a metal atom ligated to at least one oxygen atom in which a peroxide ion coordinates with the metal atom; peroxometalates are a subset of oxometalates. The term includes but is not limited to pervanadate, permolybdate, and pertungstate.

A dandruff-attenuating adhesion modulator is an agent that alters intercellular adhesion characteristics of cells, which, when added to a suitable formulation at an effective concentration and applied to the skin, attenuates the dandruff-producing process. The mechanism of attenuating the dandruff-producing process may include the disruption of dandruff flakes in that a dandruff-attenuating adhesion modulator may cause disruption of dandruff flakes into smaller, less visible pieces, but the term applies to the dandruff-producing process in the skin and not exclusively or specifically to the disruption of dandruff flakes. In a non-exclusive list, dandruff-attenuating adhesion modulators include vanadyl, vanadate and pervanadate.

The formulations, methods, and processes of the present invention can comprise, consist of, or consist essentially of the essential elements described herein, as well as any of the additional or optional ingredients, components described herein or which are otherwise effective for such use. Therefore, the inventive compositions, methods and processes include claims regarding these agents and their utility for compositions, methods and processes useful for the treatment of symptoms of dandruff.

The methods and compositions of the present invention effectively reduce or substantially reduce the average size of released flakes or the rate of release of flakes readily visible to the naked eye, or both, resulting in markedly reduced or eliminated visibility of flakes on the scalp, hair or clothes of a person with dandruff. As such, the compositions and methods of the present invention reduce or eliminate one or more symptoms of dandruff for a person desiring such treatment and may be applied to animals with similar conditions. The methods, processes and compositions of the present invention act to reduce the symptoms of dandruff as such are visible as flakes on the hair, skin or clothing of a person rather than acting solely or primarily on a microbe in the causation of dandruff flakes. During and after use of the present invention's methods, processes and compositions, a user's scalp or skin may still be colonized by the same fungus that was contributing to the triad resulting in the user's dandruff.

Treatment of dandruff by addressing the size and/or rate of shedding of the dandruff particles is a novel way of addressing a long-standing consumer problem. In this regard, the inventors have identified exemplary materials that act effectively as dandruff-attenuating adhesion modulators.

As the causative mechanism of dandruff is very complex and has not been definitively resolved after over 100 years of study, establishing precisely how a dandruff-attenuating adhesion modulator interrupts dandruff production cannot presently be established with complete certainty. Without being bound by theory, it is believed that the adhesion modulators of the present invention alter the strength of adhesion between corneoocytes in the desquamation process, slowing the process to a rate closer to normal such that corneocytes are released in smaller clusters than are typical for the dandruff-generating scalp. Also without being bound by theory, while adhesion modulators are known to alter cellular adhesion, the present invention may influence other cellular functions (human or microbe) that are significant in the causation of dandruff, which, in turn, cause a reduction in the user's symptoms of dandruff.

The inventors herein have found that an adhesion modulator (vanadate, for example) at very low concentrations in a suitable carrier formulation is surprising in its effectiveness. Indeed, the summary effect of the application of this adhesion modulator to dandruff-producing scalp is unexpected and remarkable, as the sloughing corneocytes, having lost their nuclei as they matured in the strata below are generally regarded as inert and are not expected to respond to subtle extracellular influences. The effect of the inventive compositions and methods is particularly surprising because the scientific literature discloses opposing effects of oxometalates (on nucleated cells only) including increased adhesion of desmosomes (Garrod et al, 2008) and decreased integrity of adherens junctions (Volberg et al, 1992). The effect of the adhesion modulator on the anucleate cells of the stratum corneum and stratum disjunctum may be due to changes in desmosomes (corneosomes), squamosomes, adherens junctions, or other structures including those utilized by the resident fungus for adherence. Some portion of the applied adhesion modulator may penetrate to the nucleated cells of the deeper strata and manifest the desirable effect through changes in these cells.

In typical users, the inventive methods, processes and compositions yield beneficial effects within about a week of treatment where such beneficial effects will last for about a week or more. In contrast to shampoo-based antidandruff preparations, the normal application of which results in most of the active agent being rinsed down the drain, releasing the antimicrobial agent into the environment, the use of a leave-on treatment according to the inventive method applies most of the active agent to the affected area. Use of the invention in the embodiment of a leave-on treatment also allows the user to use his or her preferred shampoo and conditioner prior to use of the invention. Use of the invention relieves or eliminates dandruff symptoms with no adverse visible changes in the skin or hair.

In separate aspects, the dandruff-attenuating adhesion modulators of the present invention comprise one or more of: vanadyl, vanadate, pervanadate, and the chemical class to which these agents belong, the oxometalates, which have been shown to have similar effects on epithelial cells (including but not limited to molybdate, permolybdate, tungstate and pertungstate).

The inventive methods comprise the step of applying to the skin or scalp one or more adhesion modulators in a carrier at a concentration that is effective and is also suitable for such application. The methods described are suitable for various embodiments of the inventive method as the application of a leave-on, a shampoo, a conditioner, a lotion, a cream, a styling composition, a gel, a spray, a tonic, or a combination thereof or other hair or skin preparation.

As a general matter, the concentrations of the adhesion modulator can be described in terms of molality (mol/kg) for a finished, applied composition. In a leave-on liquid composition, an adhesion modulator such as vanadate will be generally represented as from about 10⁻¹² mol/kg to about 10⁻³ mol/kg. While adhesion modulators in concentrations less than 10⁻⁹ mol/kg may seem too dilute to cause effects, after application of a leave-on treatment, the evaporation of liquid carrier (e.g., water or other excipient) can result in significant concentration of the adhesion modulator on the hair and skin of the user, so formulations comprising extremely dilute concentrations of an adhesion modulator may still be effective when used as leave-on treatments.

Because the oxometalate ion is the active agent in the composition as applied in the inventive method and the ion is available in a number of salts with various molecular weights, it is practical to describe the effective range of concentrations in molality of the vanadate ion or pervanandate ions. Vanadate ion, (VO₄⁻³) at about 10⁻⁹ to about 10⁻³ mol/kg is about 0.00000001% to about 0.01% of the solution by mass. Higher concentrations of adhesion modulators can be useful for shampoo or conditioner preparations wherein the preparation is rinsed from the skin after application.

### Methods

The present invention comprises methods for reducing one or more symptoms of dandruff, the inventive method comprising: applying a composition comprising a dandruff-attenuating adhesion modulator such as an oxometalate or combinations thereof, preferably an oxometalate comprising vanadium, most preferably comprising vanadate or pervanadate, to the skin or scalp of a person demonstrating the symptoms of dandruff in an amount sufficient to treat a symptom of dandruff. While the preferred adhesion modulators for this method are vanadate and pervanadate, other oxometalates and adhesion modulators are predictably effective.

In significant aspects, the inventive method may be used with any frequency found to be effective. Daily application is adequate to achieve the attenuation of a symptom of dandruff. Application to moist hair and skin may improve dispersion and evenness of application.

The adhesion modulator applied according to inventive method may also be incorporated into a gel, cream, ointment, salve, balm, lotion or powder for enhancement of efficacy of application to scalp or non-scalp skin. Optional ingredients include any cosmetically acceptable ingredients such as those found in the Personal Care Products Council International Cosmetic Ingredient Dictionary and Handbook, 13th edition, incorporated herein by reference in its entirety.

### Processes

For research use, adhesion modulators are typically packaged in quantities of a gram or greater from which quantities of a milligram or greater are dispensed for use. One aspect of the invention includes the process of manufacturing comprising a step in which an amount of an adhesion modulator is placed in a container affixed to which is a means for dispensing or applying the finished composition in an amount suitable for application according to the inventive methods. This inventive process provides novel utility by delivering an effective volume of an appropriately diluted concentration of the inventive composition to the user for application to the skin or scalp. This inventive process makes practical and accessible the dispensing of an amount of adhesion modulator that is of value to the user for the effects delivered by the inventive method. The applied volume and concentration is far less than would be practical or useful for transferring adhesion modulators except in use according to the inventive method.

Alternative embodiments of this aspect of the inventive process include packaging the adhesion modulator-containing composition as a finished composition, a liquid concentrate, a solid, a powder, a gel, a granular mixture, or as an aggregate or a semisolid to be reconstituted by the addition of a carrier at the time of use to create the applied composition. For advantages in economy and environmental sustainability, a liquid carrier such as water may be added by the user to the container prior to each use or prior to first use of the finished composition according to the inventive method.

For this purpose a liquid solution, solid, powder, gel, suspension, aggregate, granular mixture, crystal, emulsion or tablet comprising an amount of adhesion modulator sufficient to create an effective concentration for a defined user-added volume of carrier (which could range from less than about 5 milliliters for a one-time individual use to greater than about 5 liters) is packaged in a solid, semi-solid, permeable, soluble, removable or other enclosure for dilution with water or other carrier in the same enclosure or a container. In one illustrative example when sodium vanadate is used, the adhesion modulator could have a packaged weight range from about 0.00005 mg (5 ml, single use, 5×10⁻⁸ mol/kg concentration) to about 1 mg (5 liters, multi-use, 1×10⁻⁶ mol/kg concentration). Because of the very small mass of adhesion modulator required, a soluble carrier (e.g., a salt), an insoluble carrier (e.g., silica), or an added active ingredient (e.g., vitamin) may also be combined with the adhesion modulator for easier handling and measuring.

The dispensing means may include a nozzle, pump, flow-restricting cap, valve or similar device that delivers to the user's skin an amount of the finished composition having an effective concentration of an adhesion modulator. A squeeze bottle with a nozzle may deliver this amount at once or in parts to supply the entirety of an application. Alternatively, and to insure even, thorough coverage, a user may use 100 pumps or more of a spray pump or aerosol device to apply the adhesion modulator to the skin or scalp.

### Compositions

The composition applied according to the inventive method may be enhanced by the addition of any of the following: an alcohol, a skin sensate, a fragrance, a surfactant, an oxidizer, a preservative.

### Alcohols

In order to solubilize other components of the preparation, an alcohol such as ethanol or propanol may be included in the composition. Alcohols are known to form esters with vanadate; however, evaporation of the alcohol from the composition on the scalp reduces the alcohol concentration rapidly, pushing the equilibrium toward free aqueous vanadate. The alcohol may be particularly advantageous in speeding the drying of a leave-in composition, or in the case of heavier alcohols, for a desirable effect on hair luster.

### Skin Sensate

A further aspect the present invention involves combining an adhesion modulator with a skin sensate. A skin sensate in the inventive composition helps provide a sensory confirmation of the adequacy, activity and evenness of the application thereof by a user. Some non-limiting examples of skin sensates are described in U.S. Pat. Nos. 4,230,688, 4,136,163, 6,183,766 and 7,001,594 each of which are incorporated herein by reference in their entireties. Non-limiting examples of suitable sensates include butanedioic acid monomenthyl ester, camphor, carvone, cineole, clove oil, ethyl carboxamide, ethyl menthane carboxamide, eucalyptus oil, eucolytol, ginger oil, l-isopulegol, menthol, menthone glycerin acetal, menthoxy-1,2-propanediol, menthyl lactate, methyl diisopropylpropioniamide, methyl salicylate, peppermint oil, rosemary oil, trimethyl butanamide, vanillyl butyl ether or combinations thereof, concentrations of which can be included in the compositions (as measured by total weight of the adhesion modulator-containing composition) at from about 0.01% to about 2% or from about 0.01% to about 1% by total weight of the composition.

### Fragrance

A further aspect of the present invention is a composition comprising an adhesion modulator and a fragrance. Along with enhancing the consumer's perception of the composition, fragrance in the inventive composition helps provide sensory confirmation of adequacy of application and activity of the composition. Because the other components of the composition have no odor needing masking, selection of a fragrance composition is restricted only by the necessity of preserving the stability and appearance of the carrier or base and function of the active agent, a common process in consumer product development. Fragrances will often include a nonionic surfactant and must be chemically compatible with the adhesion modulator and any other additives. A list of possible fragrances is provided in U.S. Pat. Nos. 4,534,891, 5,112,688, and 5,723,420, each of which is incorporated herein by reference in their entireties. Suitable fragrances are described in Perfumes, Cosmetics and Soaps: The Raw Materials of Perfumery, 7th Edition (edited by W. A. Poucher and G. M. Howard, 1975) and The Personal Care Products Council International Cosmetic Ingredient Directory and Handbook, 13th Edition (Gottschalck and Bailey, editors). Among the fragrance choices are acacia, banana, fern, gardenia, honeysuckle, hyacinth, jasmine, lilac, lily, magnolia, mimosa, narcissus, orange blossom, orchids, vanilla, and violet. Fragrance concentrations may be present in the composition in an amount (as measured by total weight of the adhesion modulator-containing composition) of from about 0.0001% to about 2% or from about 0.001% to about 0.5% by total weight of the composition.

### Surfactant

A further aspect of the invention combines an adhesion modulator and a surfactant to provide a distinctly advantageous dandruff-attenuating adhesion modulator-containing composition. In this regard, the invention can be provided as a conditioner, shampoo or similar product. The total amount of surfactant used in the inventive composition can be from about 5% to about 70% by weight or from about 8% to about 40% or from about 10% to about 35% by weight based on the finished composition. The surfactant may be ionic, cationic, nonionic, or combinations thereof.

When an anionic surfactant is utilized, the amount of anionic surfactant in the compositions according to the invention (as measured by total weight of the adhesion modulator-containing composition) can be from about 7% to about 30% or from about 10% to about 25% or from about 12% to about 22%. When a cationic surfactant is utilized in the adhesion modulator-containing compositions, the total amount (as measured by total weight of the adhesion modulator-composition) of cationic surfactant in the composition according to the invention can be from about 0.05% to about 10% or from about 0.05% to about 5% or from about 0.1% to about 3%. When non-ionic surfactant are used in the adhesion modulator-containing compositions, the amount of surfactant can be from about 1% to about 20% or from about 2% to about 10% or from about 3% to about 7%. When an amphoteric surfactant is used in the adhesion modulator-containing compositions of the present invention, the amount of amphoteric surfactant (as measured by total weight of the adhesion modulator-containing composition) can be from about 0.5% to about 20% or from about 1% to about 10%. Examples of surfactants suitable for use in the present invention are described in U.S. Pat. Nos. 6,663,875 and 6,183,766 both incorporated herein by reference in their entireties.

### Oxidizer

An oxidizer is necessary to create or maintain the presence of a peroxometalate. The oxidizer can be present in a wide range of concentrations relative to the oxometalate, from less than 1/1000 of the oxometalate concentration to a molar equivalent or greater. When the oxidizer is a peroxide, it forms the peroxometalate from the oxometalate. A non-peroxide oxidizer may help preserve the peroxometalate, supporting a longer shelf-life, additionally acting as a preservative. Alternatively, the peroxide may be added by the user prior to the first use of the packaged product to create or increase the concentration of peroxometalate, or added in portions to an isolated portion of oxometalate prior to each use of the packaged product (multi-component packaging) to create or increase the concentration of a peroxometalate.

Because of its well-known safety profile and anti-microbial effect, hydrogen peroxide is considered the optimal oxidizer for the preferred embodiment, but other oxidizers such as calcium hypochlorite or sodium hypochlorite may be used alone or in combination. An excess of oxidizer could cause bleaching of the hair and is to be avoided.

### Preservative

The oxidizers listed above will also act as preservatives in many cases. While one or more preservatives would be a nonsensical addition to an adhesion modulator in the context of cell culture, it is a reasonable component of a topical product suitable for use in the inventive method, particularly if the applied composition has ingredients capable of supporting growth of bacteria or fungi. In addition to the oxidizers listed above, other common preservatives may be useful provided they do not interfere with the function of the dandruff-attenuating adhesion modulator. Common preservatives that may be compatible for use include citric acid, EDTA, BHA, BHT, and ascorbic acid. Other antioxidant, chelating, and antimicrobial preservatives may be found in the Handbook of Preservatives (Ash & Ash, 2004), incorporated herein by reference.

For the use of the invention, a wide variety of preparations may be used in addition to the leave-on described in the examples. Examples of further preparations include hair rinses, hair shampoos, hair conditioners, leave-on hair or scalp preparations, hair styling products, hair sprays, hair creams, hair oils, hair gels, hair foams, hair lotions, hair tonics, or solid tablets or powders.

The compositions applied according to the present invention may further comprise one or more optional components known for their use in hair care or personal care products, provided that the optional components are physically and chemically compatible with the essential components of the compositions described herein and do not otherwise unduly impair product performance.

### Carriers

In addition to the adhesion modulator active ingredients, the composition applied using the inventive method may comprise one or more carriers. A carrier may be aqueous or non-aqueous, silicone-containing or non-silicone containing and can be any carrier that is suitable, that is pharmaceutically acceptable, for application to the skin and does not unduly impact performance of the adhesion modulator.

The low concentration of the leave-on embodiment of the inventive method can present advantages over existing shampoo-type treatments (that is, those compositions that do not comprise a dandruff-attenuating adhesion modulator of the present invention). In particular, the low concentration of the adhesion modulator in the applied composition can be advantageous by providing a significant margin of safety. One of the adhesion modulators of the present invention, vanadate, contains vanadium, which is generally regarded as safe for chronic exposure (and ingestion) by humans up to 200 μg/d (Domingo, 1996).

Given the surface area of the scalp, approximately 600 cm² (0.06 m², Schwartz, 2005) and the size of the exposed surface of a cell (˜880 μm², Agache and Humbert, 2004) the number of cells exposed can be estimated (˜7×10¹¹). With 10,000 cm² (1 m²) of hair surface area in the proximal two centimeters of hair shafts (Schwartz, supra), efficiency of delivery to the scalp surface is likely to be close to 5 percent.

Application according to the inventive method of about 5 mL of a 300 nmol/kg vanadate/pervanadate solution with about 5% scalp application efficiency applies only about 4 ng of vanadium to the skin, 1/2,500 of the amount of vanadium in a typical multivitamin/mineral supplement (10 μg) and 1/50,000 of the limit suggested by Domingo (supra.) Most of the 5 ng of the applied active is likely shed as the skin cells of the stratum disjunctum are sloughed, so penetration through the skin into the general circulation is unlikely. Still more of the active agent is likely to be absorbed by post-application contact with hair or removed at a later time by the user's routine shampooing. If any of the applied material is absorbed beyond the outer skin layer, dilution in the tissues and bloodstream can render the concentration of the adhesion modulator too low to cause unintended adverse effects in a user. However, in significant aspects, the dandruff-attenuating adhesion modulator of this example is generally regarded as safe for topical use at low concentrations.

### EXAMPLES

The following examples illustrate the methods and processes of the present invention. The examples and embodiments described herein are for illustrative purposes only; various modifications or changes in light thereof will be suggested to one skilled in the art without departing from the scope of the present invention.

### Example 1

### Method

According to the inventive method, a composition comprising an adhesion modulator, as shown in Table 1 is applied to the skin or scalp in an amount sufficient to reduce a symptom of dandruff.

TABLE 1
300 nM Vanadate Leave-on treatment

| | |
|---|---|
| Sodium orthovanadate | 0.0000055% (15 μg vanadium/L) |
| Fragrance | 0.1% |
| Water | q.s. to 100% |

In this example, a 300 nM solution of an adhesion modulator, sodium vanadate, is applied twice daily to the scalp of a person with symptoms of dandruff. Table 2 reports subjective symptom scores after use of the inventive method for a week:

TABLE 2
Symptoms of Dandruff: aqueous 300
nmol/kg Vanadate/Pervanadate

| Day # | pre-treatment | 2 | 4 | 6 |
|---|---|---|---|---|
| Flaking | 100 | 100 | 10 | 0 |
| (% max) | | | | |
| Flake size | 0.2-1.0 mm | 0.2-1.0 mm | fine, 0.1-.0.2 mm | NA |
| hair shedding | 100 | 100 | 50 | 25 |
| (% max) | | | | |

Application of the composition described in Table 1 according to the inventive method reduced the dandruff flake size, the visible quantity of flakes and the rate of hair loss. By the sixth day of treatment, flakes were no longer visible and hair shedding was reduced to about one-fourth of the pre-treatment level.

### Example 2

### Process

A 100 ml bottle is filled with 300 nM vanadate in water and capped with a pump-spray device with an extended nozzle to allow direction of the spray to the scalp without saturating the hair. Upon operation of the pump-spray mechanism, approximately 0.05 ml of the solution (1.5×10⁻¹¹ mol of vanadate) is dispensed per pump, totaling about 1.5×10⁻⁹ mol for the application (100 operations of the mechanism.)

We claim:

1. A method for treating a symptom of dandruff comprising the step of applying a dandruff-attenuating adhesion modulator to the scalp or skin of a mammal having a symptom of dandruff.

2. The method of claim 1 wherein the dandruff-attenuating adhesion modulator comprises an oxometalate.

3. The method of claim 1 wherein the dandruff-attenuating adhesion modulator comprises vanadate.

4. The method of claim 1 wherein the dandruff-attenuating adhesion modulator comprises pervanadate.

5. The method of claim 3 wherein the vanadate comprises from about 10⁻¹² mol/kg to about 10⁻³ mol/kg of the applied composition.

6. The method of claim 4 wherein the pervanadate comprises from about 10⁻¹² mol/kg to about 10⁻³ mol/kg of the applied composition.

7. A process comprising
the step of placing a dandruff-attenuating adhesion modulator into a container having a volume from about 5 milliliters to about 5 liters and
the step of provisioning said container with a means of dispensing an effective amount of the dandruff-attenuating adhesion modulator by operation of said means.

8. The process of claim 7 wherein the dandruff-attenuating adhesion modulator comprises vanadate in a concentration from about 10⁻¹² mol/kg to about 10⁻³ mol/kg of the finished composition.

9. The process of claim 7 wherein the dandruff-attenuating adhesion modulator comprises pervanadate in a concentration from about 10⁻¹² mol/kg to about 10⁻³ mol/kg of the finished composition.

10. A composition comprising a fragrance and a dandruff-attenuating adhesion modulator, wherein the dandruff-attenuating adhesion modulator comprises from about 10⁻¹² mol/kg to about 10⁻³ mol/kg of the finished composition.

11. A composition comprising a surfactant and a dandruff-attenuating adhesion modulator, wherein the dandruff-attenuating adhesion modulator comprises from about 10⁻¹² mol/kg to about 10⁻³ mol/kg of the finished composition.

12. A composition comprising a skin sensate and a dandruff-attenuating adhesion modulator, wherein the dandruff-attenuating adhesion modulator comprises from about 10⁻¹² mol/kg to about 10⁻³ mol/kg of the finished composition.
